(19) United States
(12) Patent Application Publication
(10) Pub. No.: US 2008/0208191 A1
(43) Pub. Date: Aug. 28, 2008

(54) Unitary blood vessel dissector and harvester device

(75) Inventor: Randal James Kadykowski, South Lyon, MI (US)

Correspondence Address: TERUMO CARDIOVASCULAR SYSTEMS CORPORATION;ATTN: GAEL DIANE TISACK, 6200 JACKSON ROAD, ANN ARBOR, MI, 48103, US

(73) Assignee: Terumo Cardiovascular Systems Corporation

(21) Appl. No.: 11/709,950

(22) Filed: Feb. 23, 2007

(51) Int. Cl. A61B 18/14

(52) U.S. Cl. 606/46

(57) ABSTRACT

A unitary dissector/harvester device for dissecting and harvesting a vessel has an elongated dissector tool for insertion into a body having the vessel to be harvested and a harvester tool axially positioned within the dissector tool.

### CROSS-REFERENCE TO RELATED APPLICATIONS

Not applicable.

### STATEMENT REGARDING SPONSORED RESEARCH

Not applicable.

### BACKGROUND OF THE INVENTION

The present invention relates to the harvesting of blood vessels and, more particularly, to a method and apparatus for endoscopic dissection and retraction of sections of blood vessels for use as a coronary artery bypass graft.

In connection with coronary artery bypass grafting (CABG), a blood vessel or vessel section, such as an artery or vein, is “harvested” (i.e., removed) from its natural location in a patient's body and is used elsewhere in the body. In CABG surgery, the blood vessel is used to form a bypass between an arterial blood source and the coronary artery that is to be bypassed. Among the preferred sources for the vessels to be used as the bypass graft are the saphenous vein in the leg and the radial artery in the arm.

Endoscopic surgical procedures for harvesting a section of a blood vessel (e.g., the saphenous vein) subcutaneously have been developed in order to avoid disadvantages and potential complications of harvesting of the blood vessel. In the past, the harvesting was done through a continuous incision (e.g., along the leg) which exposed the full length of the desired vein section. The continuous incision had been necessary in order to provide adequate exposure for visualizing the vein and for introducing the surgical instruments to sever, cauterize and ligate the tissue and side branches of the vessel.

A more recent development has been a minimally-invasive technique that employs a small incision for locating the desired vessel and for introducing one or more endoscopic devices into the small incision. The first step was the primary dissection of the vessel in order to separate the vessel from surrounding tissues within the patient's body. The primary dissection of the vessel occurred by introduction of a dissecting instrument through the small incision to create a working cavity (i.e., a “tunnel” around all sides of the vessel) and to separate the vessel from the surrounding tissue. The dissecting instrument was then removed from the patient's body.

The second step was the removal, or harvesting of the vessel from the patient's body. The harvesting of the vessel occurred by introduction of a cutting instrument into the working cavity to sever the blood vessel from the connective tissue surrounding the vessel. The harvesting included severing the proximal and distal ends of the harvested vessel and the severing of all the side branches extending from the harvested blood vessel. The branches were then clipped and/or cauterized.

In one typical procedure, a first incision is made as the endoscopic entry site. The first incision is often located near a midpoint of the vessel being harvested, with the dissection of the vessel and the cutting of branches proceeding in both directions along the vessel from the entry site. In order to remove the desired section of the blood vessel, a second small incision, or stab wound, is made at one end thereof and the blood vessel section is ligated. A third small incision is made at the other end of the blood vessel section which is then ligated, thereby allowing the desired section to be completely removed through the first incision. Alternatively, only the first two incisions may be necessary if the length of the endoscopic device is sufficient to obtain the desired length of the blood vessel while working in only one direction along the vessel from the entry point.

Commercially available products for performing the endoscopic blood vessel harvesting procedure described above include the dissector device and the harvesting device which are sold together as the “VirtuoSaph™ Endoscopic Vein Harvesting System” from Terumo Cardiovascular Systems Corporation of Ann Arbor, Mich. Endoscopic vein harvesting systems are also shown in U.S. Pat. No. 6,660,016 to Lindsay, U.S. Pat. No. 7,077,803 to Kasahara et al., U.S. patent application publication Nos. 2005/0010242 and 2005/0192612 both in the name of Lindsay, and U.S. patent application publication numbers 2005/0154257A1, 2005/0159764A1, 2005/0148817A1, 2005/0149094A1 in the name of Kasahara et al., all of which are incorporated herein by reference in their entirety.

In the VirtuoSaph™ System and Kasahara et al. Pub. No. '764, an endoscope is used to visualize both the dissection and harvesting procedures. The endoscope image is necessary for the operator to dissect the peripheral tissue along the blood vessel.

It would be desirable to reduce the time and to simplify the procedures for both dissecting and harvesting of vessels.

### SUMMARY OF THE INVENTION

A unitary endoscopic device for dissecting and harvesting a vessel has an elongated dissector tool for insertion into a body having a vessel to be harvested, and a harvester tool axially positioned within the dissector tool. In certain embodiments, the harvester tool is coaxially displaced with respect to the dissector tool and the harvester tool is advanced through a distal end of the dissector tool. In certain other embodiments, the dissector tool is coaxially displaced with respect to the harvester tool and the dissector tool is retracted along the harvester tool to expose a distal end of the harvester tool.

An endoscope is co-axially positioned within the dissector tool in order to visualize the dissection and harvesting of the vessel.

The dissector tool includes a sheath having a dissector tip which is used for separating peripheral tissue from the vessel being harvested. In certain embodiments, the dissector tip is defined by an oblique plane extending through a distal end of the sheath. In other embodiments, the dissector tip has a conical shape that includes at least one opening to allow the harvester tool to be displaceable with respect the dissector tip.

A method of severing branches from a vessel during harvesting from a body includes inserting the endoscopic device into the body alongside the vessel, using the dissector tool to form a cavity substantially surrounding the vessel, and engaging the harvester tool to secure the vessel and to individually sever any branches attached to the vessel.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 is an exploded structure diagram showing an operation system comprising a unitary dissector/harvester device and an endoscope apparatus useful with the unitary dissector/harvester device.

FIG. 2 is a top structure diagram of a distal end of a unitary dissector/harvester device showing a harvester tool in an open and extended position in a sheath, and showing a distal end of the dissector tool where a closure thereon is in phantom.

FIG. 3 is a perspective structure diagram of a distal end of a dissector/harvester device showing a harvester tool in an extended position.

FIG. 3A is a perspective structure diagram of a distal end of a dissector/harvester device showing a harvester tool in a closed position.

FIG. 4 is a top structure diagram of a portion of a harvester V-cutter.

FIG. 5 is a cross-sectional structure diagram of a unitary dissector/harvester device.

FIG. 6 is a view taken along the line 6-6 in FIG. 5.

FIG. 7 is a perspective structure diagram of another embodiment of a distal end of a unitary dissector/harvester device showing a harvester tool in a closed and extended position in a dissector tool.

FIG. 8 is a side elevational structure diagram of the embodiment shown in FIG. 7, and showing, in phantom, a dissector tool and a harvester tool.

FIG. 9 is another perspective structure diagram of the distal end of the embodiment shown in FIGS. 7 and 8.

FIG. 10A is a side elevational structure diagram of another embodiment of a distal end of a unitary harvester/dissector device in a closed position.

FIG. 10B is an end structure diagram of the harvester/dissector device shown in FIG. 10A.

FIG. 11A is a side elevational structure diagram of another embodiment of a distal end of a unitary harvester/dissector device in an open position.

FIG. 11B is an end structure diagram of the harvester/dissector device shown in FIG. 11A.

### DETAILED DESCRIPTION OF THE PREFERRED EMBODIMENT

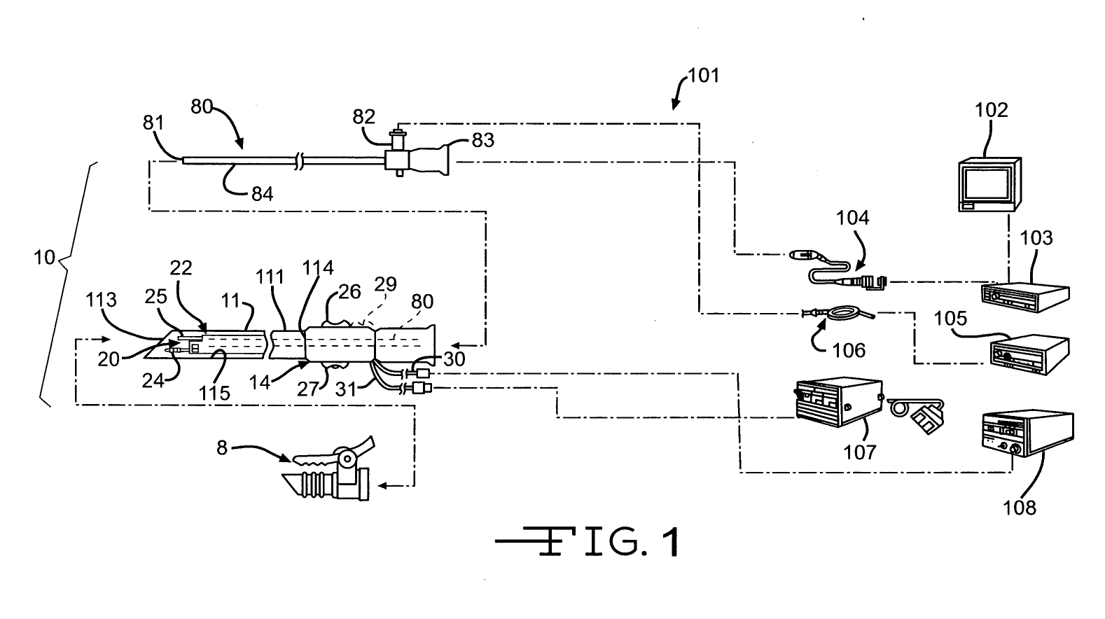

FIG. 1 is a structure diagram showing the structure of an operation system 101 useful for the above-mentioned operation. In the embodiment shown, the operation system 101 generally includes a trocar 8, a unitary dissector/harvester device 10, and an endoscope 80. The unitary dissector/harvester device 10 includes a dissector tool 11 and a harvester tool 20, as further explained below.

The operation system 101 further includes a monitor 102 as a display device; a camera control unit 103; a camera device 104; a light source device 105; a light guide cable 106; an electric knife device 107; and, a gas feed device 108.

One end of the light guide cable 106 is connected to a light guide connector portion 82 of the endoscope 80. Another end of the light guide cable 106 is connected to the light source device 105. The light from the light source device 105 is supplied to the endoscope 80 via the light guide cable 106 through which an optical fiber is inserted.

A subject is illuminated with light from a front end portion 81 of the endoscope 80. A camera head portion of the camera device 104 is connected to an eyepiece portion 83 on a base end side of the endoscope 80. The camera device 104 is connected to the camera control unit 103, and an image of the subject captured by the endoscope 80 is displayed on the screen of the connected monitor 102.

In order to view the subject, an inserting portion 84 at the front end 81 of the endoscope 80 is inserted in a proximal end of the unitary dissector/harvester device 10, as further explained below.

An insufflation tube 30 is connected to the gas feed device 108, and a gas is fed from the gas feed device 108 to the insufflation tube 30 to the unitary dissector/harvester device 10, as further explained below.

The unitary dissector/harvester device 10 has an electric cable 31 for operatively connecting the unitary dissector/harvester device 10 to the electric knife device 107.

Referring now in particular to the unitary dissector/harvester device 10, the unitary dissector/harvester device 10 generally includes the dissector tool 11 and the coaxially aligned harvester tool 20, each of which will be described separately in detail below.

The dissector tool 11 comprises an elongated sheath 111 having a dissector tip 113 at its distal end. The sheath 111 defines an inner space 115 for receiving the harvester tool 20 and the endoscope 80. The endoscope 80 is removably positioned within the inner space 115. A proximal end 114 of the sheath 111 is operatively connected to a handle 14 and receives the endoscope 80, as further explained below.

At least a portion of the dissector tip 113 is transparent in order to allow visualization of the vessel and the surrounding tissue. Upon inserting the dissecting tool 11 under the patient's skin, it is possible to obtain an image illuminated by the illuminating light from the front end portion 81 of the endoscope 80.

To begin the dissection procedure, the dissector tip 113 is inserted through the trocar 8 that has been positioned within an initial incision in the patient. In operation, the dissector tip 113 is pressed into the tissues surrounding the vessel, thereby forming a tunnel or cavity around the blood vessel. In certain embodiments, it is desired that the dissector tip 113 be pressed into the surrounding tissue generally along the direction of the vessel in order to separate the vessel from adjacent tissue without damage to the surrounding tissue.

In certain embodiments, the insufflation tube 30 may be connected to a source of gas (e.g., CO₂) for filling the cavity adjacent the vessel as the cavity is being formed. In such embodiments, the dissecting tool 11 is airtightly connected to the handle 14. The dissector sheath 111 can include one or more discharge ports or holes 135, as shown in FIG. 3, that are in communication with the inner space 115. The gas escaping from the ports 135 allows the gas to enter the forming cavity and to keep surrounding tissue from the unitary dissector/harvester device 10.

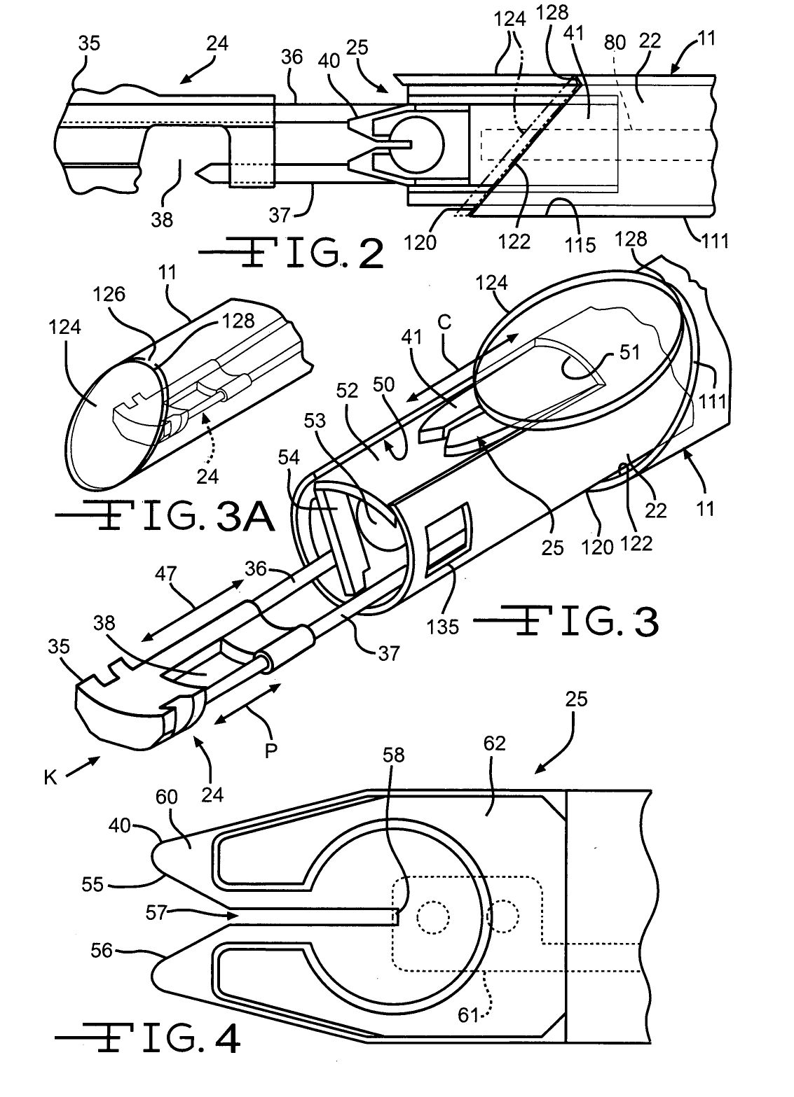

In the embodiment shown in FIGS. 1-3A and 5, the dissector sheath 111 is cylindrical-shaped at its proximal end near the handle 14. The dissector sheath 111 has a pointed shape at the tip 113. For example, best shown in FIGS. 2 and 3, the dissector tip 113 has a leading edge 120 so that an opening 122 at the distal end of the inner space 115 has an oval shape. In the embodiment shown, the opening 122 is defined by an oblique angle with respect to a plane defined by the longitudinally extending axis through the sheath 111. For example, the opening 122 can be at a 45° angle with respect to the longitudinal axis; however, it is to be understood, that in other embodiments, the angle at which the opening 122 is formed can be other than 45°.

Also, in certain embodiments, the dissector tool 11 can include a lid or closure 124 that extends over the opening 122. In such embodiments, the closure 124 is preferably transparent. The closure 124 can be operatively secured to the sheath 111 in a suitable manner. In certain embodiments, the closure 124 can be secured to the sheath 111 by a hinge 126. For example, a “living” hinge 126 can be formed on a trailing edge 128 of the opening 122.

The dissector tool 11 is advanced through the patient's tissue, forming the cavity or tunnel around the blood vessel. The dissector tip 113 is thus used to perform an initial, or blunt, dissection of the vessel. After the initial blunt dissection around the vein by the dissector tool 11, the coaxially aligned harvester tool 20 is engaged to “harvest” (or remove) the bluntly dissected vessel. As further explained in detail below, the harvester tool 20 is used to grasp the vessel being dissected and to sever any branches or connective tissue connecting to the vessel.

In the embodiment shown in FIGS. 1-3A and 5, the harvester tool 20 is coaxially displaced with respect to the dissector tool 11. In certain embodiments, the harvester tool 20 is advanced through a distal end of the sheath 111. In the embodiment shown in FIGS. 1-3A and 5, the harvester tool 20 is advanced past the distal tip 113.

Referring now, in particular, to the harvester tool 20, the harvester tool 20 is coaxially aligned within the sheath 111 of the dissector tool 11. The harvester tool 20 is operatively connected to the handle 14. The harvester tool 20 includes a vessel keeper (V-keeper) 24 for retaining the vessel being dissected and a vessel cutter (V-cutter) 25 for severing and/or cauterizing all branches attached to the vessel. In the embodiment shown, the V-keeper 24 is advanced and retracted by a V-keeper button 26 on the handle 14. The V-cutter 25 is extended or retracted by manipulating a V-cutter extender button 27 on the handle 14.

Also, in certain embodiments, as generally shown by referring to FIGS. 3 and 3A along with FIG. 5, an endoscope wiper lever 28 is provided on handle 14 for controlling a wiper 54 that clears the end of the endoscope 80 when the endoscope optics become covered by material in the body cavity.

The handle 14 can be operatively attached to the electric cable 31 at one end for connecting to a source of high frequency voltage, and includes conductors for supplying the voltage to electrodes on V-cutter 25.

In certain embodiments, as shown in FIG. 2, the V-keeper 24 and the V-cutter 25 of the harvester tool 20 are in a generally parallel relationship to each other within the dissector sheath 111. The V-keeper 24 includes a guide 35 mounted to a support tool 36 and a movable pin 37. The vein or other vessel to be harvested is maneuvered into an opening 38 and then the V-keeper buttons 26 on the handle 14 are manipulated to extend the movable pin 37 to close the opening 38 and thereby retain the vessel. The V-cutter 25 includes a V-tip 40 mounted to an extendable guide 41 that is manipulated by the V-cutter button 27 on the handle 14.

In use, when the vessel is being harvested, the vessel is retained within the opening 38 in the V-keeper 24, which, in turn, is positioned within the tunnel or cavity that had previously been created during the blunt dissection. The V-cutter 25 is then moved to an extended position (not shown) for extending toward any vessel branches that might be attached to the vessel. The V-cutter 25 is then repeatedly engaged for severing and cauterizing each branch to prepare the vessel for removal.

FIG. 3 shows the distal end of harvester tool 20 as extending from the dissector tool 11 in greater detail. The V-keeper 24 is longitudinally extendable as shown by arrow K while the movable pin 37 is independently longitudinally extendable as shown by arrow P. As shown in FIG. 3, the movable pin 37 has been extended to the closed position used for maintaining the vessel being harvested within the opening 38.

The V-cutter 25 is longitudinally extendable in the directions shown by arrow C. FIGS. 2, 3, 3A and 5 show the V-cutter 25 retracted to its inward position. While the embodiments shown in FIGS. 2 and 3 include an elongated sleeve 22 that contains both the V-keeper 24 and the V-cutter 25, it is to be understood that the harvester tool 20 can be coaxially positioned within the sheath 111 of the dissector tool 11 without first being axially aligned within the sleeve 22. For the embodiment shown in FIGS. 2 and 3, however, the sleeve 22 is shown. As such, the sleeve 22 has a notch 50 with a terminal edge 51 that exposes the V-cutter 25 prior to the V-cutter 25 being extended further than the end of sleeve 22. A guard piece 52 is provided beneath the V-cutter 25. A lens portion 53 at the end of the endoscope 80 is shown positioned near the distal end of sleeve 22.

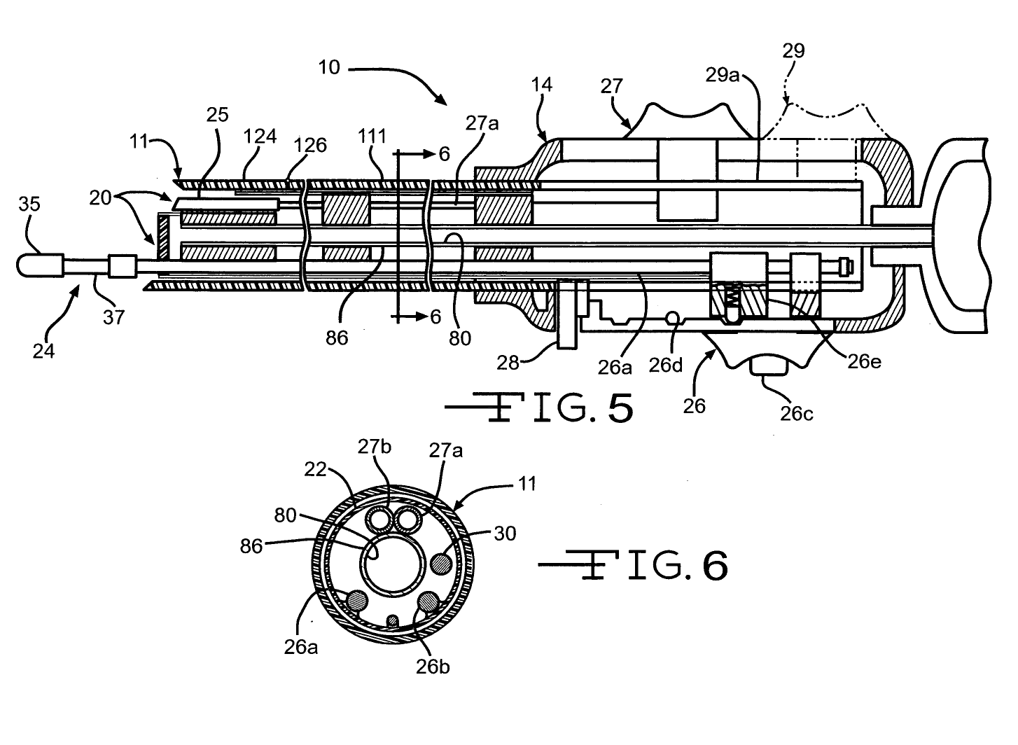

In certain embodiments, the wiper 54 is mounted on a wiper shaft 54a, as best seen in FIG. 6, for pivoting over the lens 53, as controlled by the lever 28, to wipe away debris from the lens 53.

FIG. 4 shows the V-cutter 25 in greater detail. The V-cutter 25 includes a V-tip 40 that has angled surfaces 55 and 56 for guiding a blood vessel into a longitudinal slit 57 having a terminus 58. An insulating base member 60 carries an applying electrode 61 on one side and a feedback electrode 62 on the opposite side. When the blood vessel to be severed is passing perpendicularly through longitudinal slit 57, a high frequency alternating voltage across electrodes 61 and 62 (from a generator, not shown) generates localized heating which severs and cauterizes the vessel.

During an endoscopic procedure to harvest the blood vessel, the harvester tool 20 is axially displaced from the sheath 111. The V-cutter 25 is extended and the electrodes are energized (e.g., by a foot pedal (not shown) operated by a surgeon) to individually sever the branches from the blood vessel. The V-cutter 25 is then retracted to a position longitudinally inward from its cutting position while maintaining the V-keeper 24 remains in the extended and closed position.

Referring again to FIG. 5, in particular, one embodiment of a means for axially displacing the harvester tool 20 with respect to the dissector tool 11 is shown. In the embodiment shown in FIG. 5, the harvester tool 20 is displaced in a longitudinal direction out from the sheath 111 of the dissector tool 11. Again, it is to be understood that, in other embodiments, other means are suitable for axially displacing either the harvester tool 20 or the dissector tool 11 with respect to each other.

In the embodiment shown in FIG. 5, a tube member 86 forms an endoscope-inserting channel within the sheath 111 along the axial direction of the dissector/harvester device 10. The tube member 86 extends from a base end of the handle 14 to the distal end of the sheath 111. Within the handle 14, the V-cutter 25 is connected to the V-cutter button 27 by bipolar shafts 27a and 27b (as best seen in the cross-sectional view shown in FIG. 6). The V-cutter button 27 advances and returns along the longitudinal axis of the handle 14. Then, the advancing and returning force is transmitted to the V-cutter 25 via the bipolar shafts 27a and 27b.

Similarly, within the handle 14, the V-keeper 24 is connected to the V-keeper button 26 by V-keeper, shaft 26a and V-keeper locking shaft 26b. The V-keeper button 26 advances and returns along the longitudinal direction and the advancing and returning force is transmitted to the V-keeper 24 via the V-keeper shaft 26a and V-keeper locking shaft 26b.

For ease of illustration, the embodiment shown in FIG. 5 also can include a means 29 for axially displacing the dissector tool 11 with respect to the harvester tool 20. It should be understood, however, that in certain embodiments, both the harvester tool 20 and the dissector tool 11 do not both need to be axially moveable. That is, in certain embodiments, the harvester tool 20 is axially extendable beyond the distal tip 113 of the sheath 111 of the dissector tool 11. In other embodiments, the sheath 111 of the dissector tool 11 is retractable so that the harvester tool 20 is exposed.

In certain embodiments, the harvester tool 20 is coaxially displaced with respect to the dissector tool 11 and the harvester tool 20 is advanced through a distal end of the dissector tool 11. In certain other embodiments, the dissector tool 11 is coaxially displaced with respect to the harvester tool 20 and the dissector tool 11 is retracted along the harvester tool 20 to expose a distal end of the harvester tool 20.

Referring again to FIG. 5, within the handle 14, the sheath 111 is connected to a dissector button 29 by a dissector shaft 29a. The dissector button 29 advances and returns along the longitudinal direction and then the advancing and returning force is transmitted to the dissector sheath 113 via the dissector shaft 29a. In other embodiments, the dissector button 29 can be connected directly to a proximal end 112 of the sheath 111 and can be operated to advance and/or retract the sheath 111.

In the embodiment shown in FIG. 5, the V-keeper button 26 and the V-keeper shaft 26a and V-keeper locking shaft 26b are integrally moved by a clicking mechanism 26b. When the clicking mechanism 26b is at any of three clicking grooves 26d in the handle 14, the V-keeper button 26 and the V-keeper shafts 26a and 26b are stably kept. The V-keeper button 26 is detachably connected to a locking lever 26e. When a locking button 26f is pressed down, the V-keeper button 26 is separated from the locking lever 26e. The locking lever 26e is connected to the locking shaft 26b. The locking lever 26e advances and returns in the separating state from the V-keeper button 26, thereby securing the vessel in the closed space 38 (refer to FIGS. 2 and 3). As mentioned above, referring to FIGS. 2 and 3, the V-keeper button 26 advances and returns, thereby advancing and returning the V-keeper 24 at its distal end.

Referring now to FIGS. 7-9, another embodiment of a unitary dissector/harvester device 200 is shown. For ease of illustration, similar elements will retain the same reference numerals. Referring now in particular to the unitary dissector/harvester device 200, the unitary dissector/harvester device 200 generally includes a dissector tool 210 and a coaxially aligned harvester tool 220, each of which will be described separately in detail below.

The dissector tool 210 comprises an elongated sheath 211 having a dissector tip 213 at its distal end 212. The sheath 211 defines an inner space 215 for receiving the harvester tool 220. A proximal end 214 of the sheath 211 is operatively connected to a handle (not shown) and receives an endoscope 80.

At least a portion of the dissector tip 213 is transparent in order to allow visualization of the vessel and the surrounding tissue. Upon inserting the dissecting tool 210 under the patient's skin, it is possible to obtain an image illuminated by the illuminating light from a front end portion 81 of the endoscope 80.

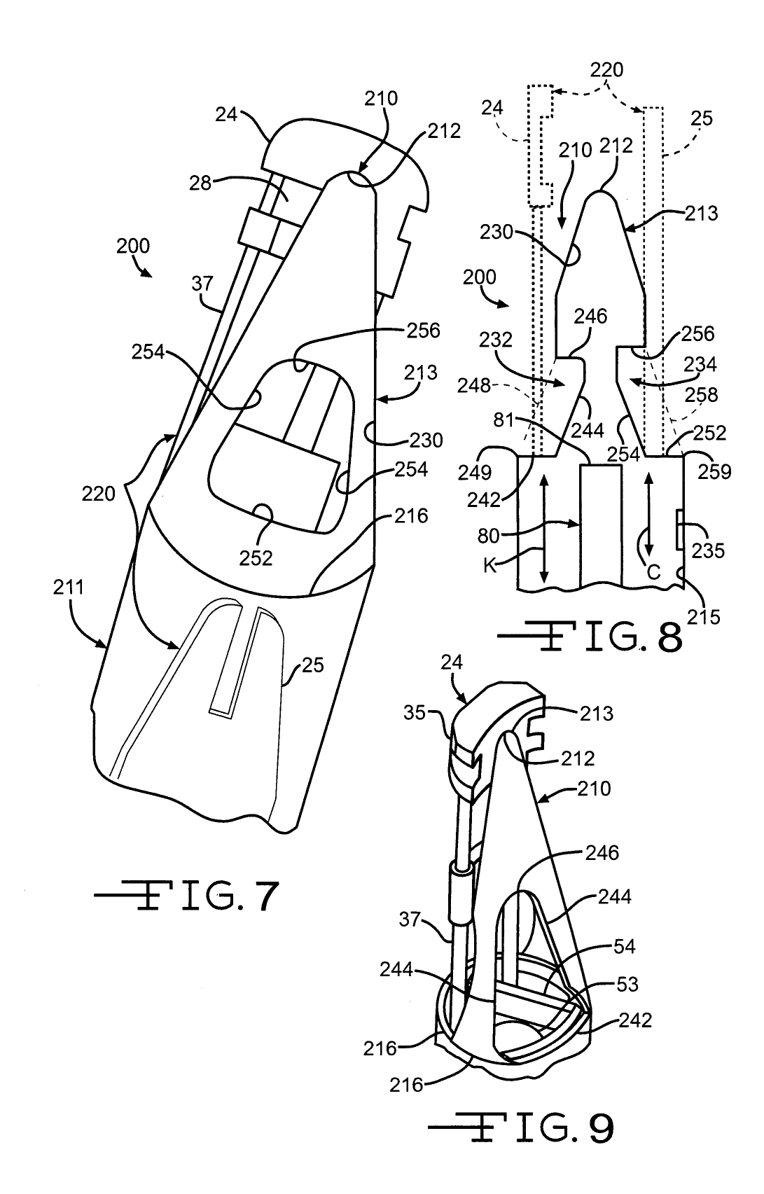

In the embodiment shown in FIGS. 7-9, the dissector sheath 211 is cylindrical-shaped at its proximal end near the handle and has a generally conical shaped tip 213. The dissector tip 213 has a tapered edge 230 that includes a first opening 232 and a second opening 234. The first opening 232 is configured to allow the V-keeper 24 to pass therethrough. Likewise, the second opening 234 is configured to allow the V-cutter 25 to extend therethrough. While the first and second openings 232 and 234 can have generally similar dimensions, other configurations are also possible. In the embodiment shown in FIGS. 8 and 9, for example, the first opening 232 has a generally trapezoidal-shaped perimeter. The first opening 232 has a base 242 that circumferentially extends around a proximal end 216 of the tip 213. Opposing sides 244 terminate at a top 246 that is adjacent to the distal end of the tip 213.

The second opening 234 has a base 252 that circumferentially extends around a proximal end 216 of the tip 213. Opposing sides 254 terminate at a top 256 that is adjacent to the distal end 212 of the dissector tip 213.

Also, in certain embodiments, as shown in phantom in FIG. 8, the dissector tip 213 can include a first lid or closure 248 that extends over the first opening 232. In certain embodiments, the closure 248 can be transparent. The closure 248 can be operatively secured to the sheath 211 in a suitable manner. In certain embodiments, the closure 248 can be secured to the sheath 211 by a hinge 249 that extends along the base 242 of the first opening 232. For example, a “living” hinge 249 can be formed along at least a part of the base 242 of the first opening 232.

Similarly, in certain embodiments, also as shown in FIG. 8, the dissector tip 213 can include a second lid or closure 258 that extends over the second opening 234. The closure 258 can be operatively secured to the sheath 211 in a suitable manner. In certain embodiments, the closure 258 can be secured to the sheath 211 by a hinge 259 that extends along the base 252 of the second opening 234. For example, a “living” hinge 249 can be formed along at least a part of the base 252 of the second opening 234.

In certain embodiments, the dissector sheath 211 can include one or more discharge ports or holes 235 that are in communication with an inner space 215 of the sheath 211 to allow gas to enter the forming cavity and to keep surrounding tissue from the unitary dissector/harvester device 200. In other embodiments, the holes 235 are not needed, and the gas can escape into the surrounding tissue though gaps defined by the perimeters of the openings 232 and 234 and the corresponding closures 248 and 258, respectively.

In a similar manner to the above-described procedure, to begin the dissection procedure, the dissector tip 213 is inserted through the trocar (not shown) that has been positioned within an initial incision in the patient. In operation, the dissector tip 213 is pressed into the tissues surrounding the vessel, thereby forming a tunnel or cavity around the blood vessel. In certain embodiments, it is desired that the dissector tip 213 be pressed into the surrounding tissue generally along the direction of the vessel in order to separate the vessel from adjacent tissue without damage to the surrounding tissue.

In certain embodiments, the insufflation tube (not shown) may be connected to a source of gas (e.g., CO₂) for filling the cavity adjacent the vessel as the cavity is being formed. In such embodiments, the dissector tool 210 is airtightly connected to the handle.

The dissector tool 210 is advanced through the patient's tissue, forming the cavity or tunnel around the blood vessel. The dissector tool 210 is thus used to perform an initial, or blunt, dissection of the vessel.

After the initial blunt dissection around the vein by the dissector tool 210, the coaxially aligned harvester tool 220 is engaged to “harvest” or remove the bluntly dissected vessel. The harvester tool 220 is engaged to grasp the vessel being dissected and to sever any branches or connective tissue connecting to the vessel.

The harvester tool 220 is coaxially displaced from the dissector tool 210 through the first opening 232. In the embodiment shown in FIGS. 7-9, the harvester tool 20 is advanced past the distal end 212 of the dissector tip 213.

The harvester tool 20 is coaxially aligned within the sheath 211 of the dissector tool 210. As described above, and shown in phantom in FIG. 8, the harvester tool 20 includes the V-keeper 24 and the V-cutter 25. In the embodiment shown, the V-keeper 24 is advanced and retracted through the first opening 232 and the V-cutter 25 is extended or retracted through the second opening 234.

In certain embodiments, as shown in FIGS. 7-9, the V-keeper 24 and the V-cutter 25 of the harvester tool 20 are in a generally parallel relationship to each other within the dissector sheath 211. The V-keeper 24 is longitudinally extendable in the directions shown by arrow K and the V-cutter 25 is longitudinally extendable in the directions shown by arrow C.

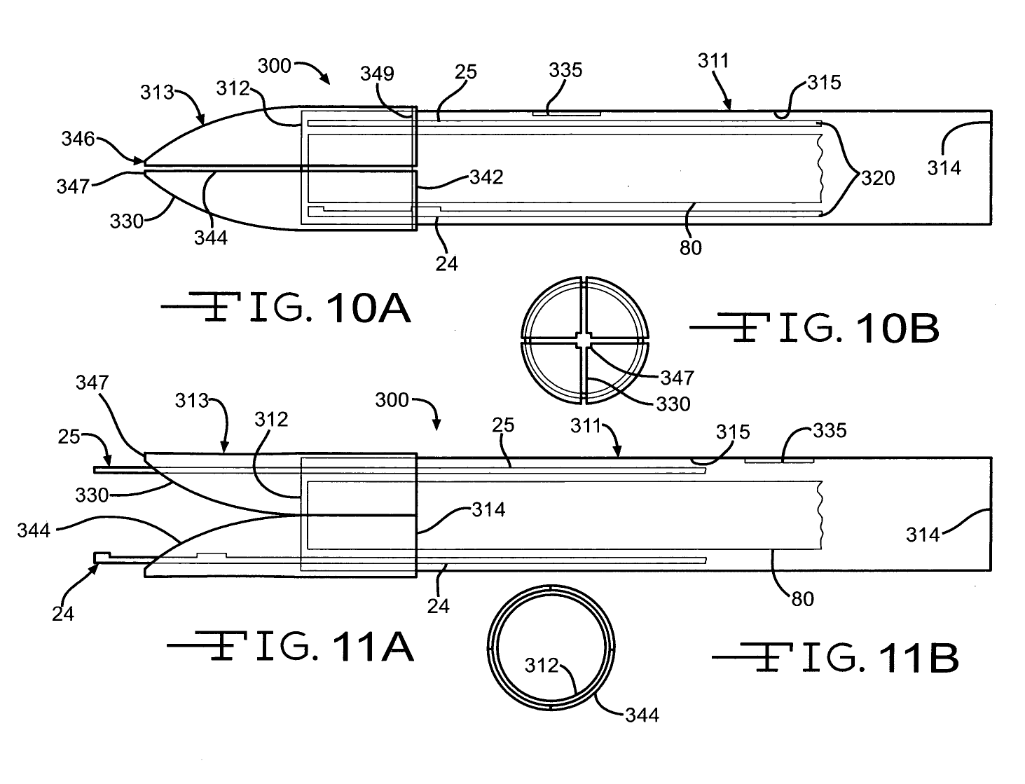

Referring now to FIGS. 10A and 10B and 11A and 11B, another embodiment of a unitary dissector/harvester device 300 is shown. For ease of illustration, similar elements will retain the same reference numerals. Referring now in particular to the unitary dissector/harvester device 300, the unitary dissector/harvester device 300 generally includes a dissector tool 310 and a coaxially aligned harvester tool 320.

The dissector tool 310 comprises an elongated sheath 311 having a dissector tip 313 at its distal end 312. The sheath 311 defines an inner space 315 for receiving the harvester tool 320. A proximal end 314 of the sheath 311 is operatively connected to a handle (not shown) and receives an endoscope.

Upon inserting the dissector tool 310 under the patient's skin, it is possible to obtain an image illuminated by the illuminating light from a front end portion of the endoscope.

In the embodiment shown in FIGS. 10 and 11, the dissector sheath 311 is cylindrical-shaped at its proximal end near the handle and has a generally conical shaped tip 313.

The dissector tip 313 has several circumferentially extending closures 330. As best seen in the embodiment shown in FIG. 10B, the dissector tip 313 includes four (4) generally triangularly shaped closures 330. The closures 330 are configured to allow the harvester tool 320 to pass therethrough. While there are four closures 330 shown that have generally similar dimensions, in other embodiments, other numbers and configurations of closures 330 are also possible. In the embodiment shown in FIGS. 10 and 11, for example, the closures 330 generally have a triangular perimeter such that the closure itself has a quarter conical shape. In the embodiment shown, each closure 330 has a base 342 that circumferentially extends around a portion of a proximal end 316 of the tip 313. Opposing sides 344 terminate at a top 346 that is adjacent to the distal end of the tip 313.

Also, in certain embodiments, as shown in FIG. 10B, the top 346 of the closure 330 can define an opening 347 such that light and/or images can be sent and received through the endoscope 80. In such embodiments, it is not necessary that the tip 313 be made of a transparent material.

The closures 330 can be operatively secured to the sheath 311 in a suitable manner. In certain embodiments, the closure 330 can be secured to the sheath 311 by a hinge 349 that extends along the base 342 of the tip 313. For example, a “living” hinge 349 can be formed along at least a part of the base 342. In such embodiments, the hinge 349 provides sufficient flexibility to the closure 330 so that advancement of the harvester tool 320 through the tip 313 pushes the closures 330 to an open position, as schematically illustrated in FIGS. 11A and 11B.

In other embodiments, the tip 313 can be fixed to the distal end of the sheath 311 and the closures 330 can have sufficient flexibility that the closures 330 are moved in a generally radially outward direction as the harvester tool 320 is advanced in a longitudinal direction through the dissector tip 313, as schematically illustrated in FIG. 11A.

In certain embodiments, the dissector sheath 311 can include one or more discharge ports or holes 335 that are in communication with the inner space 315 of the sheath 311 to allow gas to enter the forming cavity and to keep surrounding tissue from the unitary dissector/harvester device 310. In other embodiments, the holes 335 are not needed, and the gas can escape into the surrounding tissue though gaps defined by spaces between adjacent closures 330.

In a similar manner to the above-described procedure, to begin the dissection procedure, the dissector tip 313 is inserted through the trocar (not shown) that has been positioned within an initial incision in the patient. In operation, the dissector tip 313 is pressed into the tissues surrounding the vessel, thereby forming a tunnel or cavity around the blood vessel. In certain embodiments, it is desired that the dissector tip 313 be pressed into the surrounding tissue generally along the direction of the vessel in order to separate the vessel from adjacent tissue without damage to the surrounding tissue.

In certain embodiments, the insufflation tube (not shown) may be connected to a source of gas (e.g., CO₂) for filling the cavity adjacent the vessel as the cavity is being formed. In such embodiments, the dissecting tool 311 is airtightly connected to the handle.

The dissector tool 311 is advanced through the patient's tissue, forming the cavity or tunnel around the blood vessel. The dissector tool 311 is thus used to perform an initial, or blunt, dissection of the vessel.

After the initial blunt dissection around the vein by the dissector tool 311, the coaxially aligned harvester tool 320 is engaged to “harvest” or remove the bluntly dissected vessel. The harvester 320 is engaged to grasp the vessel being dissected and to sever any branches or connective tissue connecting to the vessel.

The harvester tool 320 is coaxially aligned within the sheath 311 of the dissector tool 311. The harvester tool 320 is coaxially displaced from the dissector tool 311 through the dissector tip 313. In the embodiment shown in FIGS. 10A-11B, the harvester tool 320 is advanced past the distal end 312 of the dissector tip 313.

In certain embodiments, the dissector/harvester device 10 can be included in a kit along with a trocar in order to make both the dissector/harvester device 10 and/or the trocar easy to use and dispose in a safe manner.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

What is claimed is:

1. A unitary endoscopic device for dissecting and harvesting a vessel comprises an elongated dissector tool for insertion into a body having a vessel to be harvested, and a harvester tool axially positioned within the dissector tool.

2. The device of claim 1, wherein the harvester tool is coaxially displaced with respect to the dissector tool.

3. The device of claim 2, wherein the harvester tool is advanced through a distal end of the dissector tool.

4. The device of claim 1, wherein the dissector tool is coaxially displaced with respect to the harvester tool.

5. The device of claim 4, wherein the dissector tool is retracted from the harvester tool.

6. The device of claim 1, further including an endoscope co-axially positioned within the dissector tool.

7. The device of claim 1, wherein the dissector tool includes a sheath having a dissector tip, the dissector tip being configured for separating peripheral tissue from the vessel being harvested.

8. The device of claim 7, wherein the dissector tip includes at least one closure at a distal end thereof.

9. The device of claim 8, wherein the dissector tip is defined by an oblique plane extending through a distal end of the sheath.

10. The device of claim 7, wherein the dissector tip has a conical shape, wherein the conical shape includes at least one opening configured to allow the harvester tool to be displaceable with respect the dissector tip.

11. The device of claim 10, wherein the harvester tool is advanced past the distal tip.

12. The device of claim 1, wherein the harvester tool comprises:
i) a cutter longitudinally displaceable with respect to the dissector tool toward a cutting position for cutting a branch of a vessel being harvested; and,
ii) a keeper longitudinally displaceable with respect the dissector tool toward a keeping position for securing the vessel being harvested while the branch is being cut.

13. The device of claim 12, wherein the cutter comprises a cutter tip and a longitudinal slit for receiving a branch to be cut, the cutter further comprising electrodes adjacent to the longitudinal slit for being electrically energized to cut and cauterize the branch.

14. The device of claim 1, wherein the dissector tool defines an inner space configured to receive the harvester tool and an endoscope.

15. A method of severing branches from a vessel during harvesting from a body by a unitary endoscopic device, the device having an elongated dissector tool for insertion into the body having the vessel to be harvested, and a harvester tool axially positioned within the dissector tool, the method comprising the steps of:
inserting the endoscopic device into the body alongside the vessel to form a cavity substantially surrounding the vessel; and,
engaging the harvester tool to secure the vessel and to individually sever any branches attached to the vessel.

16. The method of claim 15, wherein the harvester tool is coaxially displaced with respect to the dissector tool.

17. The method of claim 16, wherein the harvester tool is advanced through a distal end of the dissector tool.

18. The method of claim 15, wherein the dissector tool is coaxially displaced with respect to the harvester tool.

19. The method of claim 18, wherein the dissector tool is retracted from the harvester tool.

20. A method of severing branches from a vessel during harvesting from a body by a unitary endoscopic device, the device having an elongated dissector tool for insertion into the body having the vessel to be harvested, and a harvester tool positioned within the dissector tool; the harvester tool comprising i) a cutter longitudinally displaceable with respect to the dissector tool toward a cutting position for cutting a branch of a vessel being harvested; and, ii) a keeper longitudinally displaceable with respect the dissector tool toward a keeping position for securing the vessel being harvested while the branch is being cut;
the method comprising the steps of:
inserting the endoscopic device into the body alongside the vessel to form a cavity substantially surrounding the vessel;
extending the keeper to secure the vessel;
extending the cutter and energizing the electrodes to individually sever any branches attached to the vessel; and,
retracting the cutter to a position longitudinally inward from the cutting position while maintaining the vessel within the keeper.
